(19) United States
(12) Patent Application Publication
(10) Pub. No.: US 2024/0009122 A1
(43) Pub. Date: Jan. 11, 2024

(54) Small Molecule Formulation

(71) Applicant: Dialectic Therapeutics, Inc., Dallas, TX (US)

(72) Inventors: Garry R. SMITH, Dallas, TX (US); Mohammad Saiful ISLAM, Dallas, TX (US); Andrew Xian CHEN, Dallas, TX (US); Gary T. ELLIOTT, Dallas, TX (US)

(21) Appl. No.: 18/035,923

(22) PCT Filed: Nov. 10, 2021
(86) PCT No.: PCT/US2021/058843

§ 371 Date: May 8, 2023

Related U.S. Application Data
(60) Provisional application No. 63/112,132, filed on Nov. 10, 2020.

(51) Int. Cl. A61K 9/107; A61K 31/496; A61K 47/14; A61K 47/34; A61K 47/24; A61K 47/02; A61K 47/26

(52) U.S. Cl. CPC A61K 9/1075; A61K 31/496; A61K 47/14; A61K 47/34; A61K 47/24; A61K 47/02; A61K 47/26

(57) ABSTRACT

A pharmaceutically nanoemulsion formulation of Compound 1 is disclosed along with methods for making the same. An example embodiment of the nanoemulsion of Compound 1 is comprised of lecithin, medium chain triglycerides (MCT), sucrose, EDTA and water.

### RELATED APPLICATION

This application claims the benefit of U.S. Provisional Application No. 63/112,132, filed Nov. 10, 2020, which is incorporated by reference herein for all purposes.

### FIELD OF INVENTION

The present invention relates to formulations of small molecule drugs and methods for preparing and using the same.

### BACKGROUND OF INVENTION

An emulsion, generally, is a dispersion of two immiscible liquids. One of the liquids forms a dispersed phase, while the other liquid forms the dispersing medium. As such the dispersed phase is suspended in droplet form within the dispersing medium.

Emulsions may be generally categorized by the size of droplets of the dispersed phase within the dispersing medium. Nanoemulsions are distinguished from macroemulsions by emulsion droplet size. Nanoemulsions being emulsions with droplets in the nanometer size range. Macroemulsions being emulsions with larger droplet sizes. Typical droplet sizes of a nanoemulsion may range from as small as 10 nm to as large as 200 nm or more. A reference to nanoemulsion droplet sizes herein refers to an average droplet size of the nanoemulsion.

Nanoemulsions have proven useful in drug delivery and formulation. In particular, nanoemulsions are useful in formulating drugs that are hydrophobic or that present bioavailability or metabolism problems. Hydrophobic compounds are generally insoluble in water-based formulations and other highly polar solvent mediums. In order to allow for proper drug delivery, it is typically preferred that the drug component be dissolved in the formulation medium. Water or highly polar solvent-based formulations are also necessary to ensure biocompatibility of the formulation. Since hydrophobic drug compounds do not readily dissolve in water or other suitably biocompatible solvents, the formulation of highly hydrophobic drug compounds in suitably biocompatible solvents presents challenges.

The use of nanoemulsion formulations is one approach for solving formulation issues of hydrophobic drug compounds. Generally, the drug compound is dissolved in a solvent or medium in which it is soluble, even though that solvent does not present an ideal biocompatible solvent for drug formulation. The resulting drug solution is mixed with a biocompatible solvent. The resulting mixture is stabilized by an emulsifier to create droplets of the solvent used to dissolve the drug compound suspended within the biocompatible solvent. This allows for dissolution of the drug compound within the formulation, while ensuring that the primary solvent of the formulation is suitably biocompatible.

Nanoemulsions, and emulsions in general, may be classified into oil in water emulsions or water in oil emulsions. An oil in water emulsion has a lipophilic dispersed medium, such as an oil or hydrophobic nonpolar solvent, suspended in a hydrophilic dispersing medium, such as water or highly polar solvents. A water in oil emulsion is one in which the hydrophilic solvent, such as water, is the dispersed medium and the hydrophobic solvent is the dispersing medium. When used to formulate a pharmaceutically active compound, oil in water emulsions is preferred. The hydrophobic dispersed medium dissolves and disperses water insoluble pharmaceutically active compounds, while the hydrophilic dispersing medium ensures biocompatibility of the formulation. For injectable formulations, the oil in water emulsions is more common, where oil droplets ranging from 50 to 5000 nm remain suspended in the aqueous phase. For example, injectable emulsion formulations such as DIPROVAN® (Propofol injection) and INTRALIPID® (Intravenous fat emulsion) contain droplet size of about 150-500 nm diameter.

In order to successfully create a stable nanoemulsion, the components of the formulation must be carefully chosen for the particular application and desired properties. While it may be possible that a suitable dispersed phase for dissolving a drug compound and suitable dispersing medium may alone be formed into a nanoemulsion, this is often not the case. It is very common to have a need to add multiple components to the drug formulation to encourage and promote suitable nanoemulsion formation, formulation stability, and suitability for use as a pharmaceutical delivery vehicle. Generally, these include additives such as lipids, salts, oils, surfactants, cosolvents, salts, pH modifiers, buffers, etc. Many of these types of components are added to a nanoemulsion formulation to promote suitable formation of a nanoemulsion between the dispersed phase and the dispersing medium.

The desired properties of a nanoemulsion formulation depend on the particular intended application and use of the formulation. Target physical properties of a nanoemulsion formulation that are often important are drug concentration; drug stability; nanoemulsion droplet size; pH; appearance, dilutability with biocompatible solvents and fluids; osmolarity; zeta potential; filterability; viscosity, syringeability, physical stability of nanoemulsion, etc.

Therefore, nanoemulsion formulation development for a particular active pharmaceutical involves intensive research regarding combinations of dispersing mediums, dispersed mediums, and additives to achieve the desired results. This includes, but is not limited to, consideration of additive types and properties; physical and chemical properties of the active pharmaceutical, chemical stability of the components of the emulsion, physical stability of the emulsion, and intended mode of drug delivery.

Since nanoemulsions are highly dispersed emulsions with nanometer ranged droplets finely dispersed within a dispersing medium, nanoemulsions provide benefits over typical macro emulsions. These include significant increase in surface area with increased dispersion of a drug compound within a formulation; decreases in cloudiness; decreases in viscosity; decreases in other undesirable physical formulation appearance properties; and the ability to finely disperse a drug compound within a suitable biocompatible solvent. In addition, nanoemulsions are thermodynamically and kinetically more stable than macroemulsion. Once formed, the nano sized droplets of dispersed medium in a nanoemulsion are less prone to undergo aggregation and agglomeration with each other, as compared to macro emulsions. In addition, nanoemulsions, due to the decreased droplet size have a higher interfacial area to volume ratio which aids in dissolution and distribution of poorly water-soluble pharmaceutical actives.

Nanoemulsions may be formed from a number of methods including high pressure homogenization, microfluidization, ultrasonication, transitional phase inversion, catastrophic phase inversion, and self-nanoemulsification. Each of these methods usually begins with the formation of a course emulsion, except self-nanoemulsification. Subjecting the course emulsion to one of these processes results in reducing emulsion droplet sizes into the nanometer range. Each of these methods is briefly described below.

High pressure homogenization uses pressure force to disrupt course emulsion droplets into nanometer sized droplets. In general, this method uses pressure to force a course emulsion through a constricting orifice or multiple constricting orifices. The combination of several forces, such a turbulence, hydraulic shear, and cavitation, act on the course emulsion to create droplets in the nanometer range.

Microfluidization forms nanoemulsions by forcing the mixing of a course emulsion on a micro scale. The course emulsion is forced into a series of micro sized channels, which force the components of the emulsion to interact on a micro mixing scale. Formation of nanometer sized droplets is further promoted by collision of multiple fluid streams through the micro channels. Shearing, cavitation, and impact forces result in the formation of a nanoemulsion.

Ultrasonication also form nanoemulsions. In this method, the course emulsion is subjected to high intensity ultrasonic waves. This results in the formation of nanometer range droplets primarily by cavitation and turbulence forces resulting from the ultrasonic waves.

Phase inversion nanoemulsion formation techniques are low energy formation methods, as compared to high pressure homogenization, microfluidization, and ultrasonication. Phase inversion nanoemulsion formation relies on spontaneous changes in a surfactant in the formulation that occur upon shifts in parameters such a temperature and composition. There are many subtypes of phase inversion. Generally, they all use spontaneous changes in the surfactant to effect emulsion droplet sizes to create droplets with the nanometer range.

A nanoemulsion may also be formed through self-emulsification. Self-emulsification occurs when the components of the formulation inherently form an emulsion with nanometer ranged droplet sizes. However, due the various desired physical and chemical properties of drug formulations, it is relatively rare that a suitable nanoemulsion, having the desired properties, will form through self-emulsification.

### SUMMARY

One aspect of the invention pertains to a nanoemulsion formulation of Compound 1, in which Compound 1 is dissolved in a non-polar dispersed medium that is suspended in droplet form in an aquatic or polar water miscible dispersing medium. The formulation may contain formulation additives to improve the formulation physical and chemical properties; facilitate dissolution, distribution, and drug delivery of Compound 1; and to optimize nanoemulsion properties such as drug loading, droplet size, zeta potential, osmolarity, pH, physical and stability, etc.

Another aspect of the invention includes methods of making a nanoemulsion formulation of Compound 1 as described.

### BRIEF DESCRIPTION OF DRAWING

FIG. 1 is a flow diagram of one embodiment of a method for making a nanoemulsion formulation of Compound 1.

### DETAILED DESCRIPTION

The following disclosure concerns a nanoemulsion formulation and a method of forming a nanoemulsion formulation of Compound 1 or of a pharmaceutically acceptable salt of Compound 1.

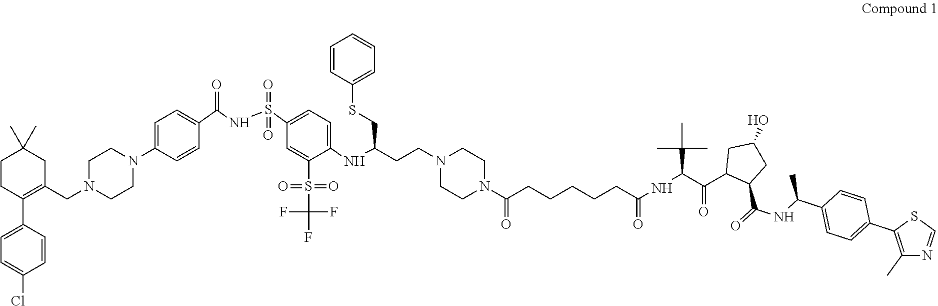 [SMILES: Cc1ncsc1-c1ccc([C@H](C)NC(=O)[C@@H]2C[C@@H](O)CC2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]

In another aspect, the disclosure includes methods for formation of a nanoemulsion formulation of a Compound 1 or of a pharmaceutically acceptable salt of Compound 1.

While Compound 1 is an example of a small molecule with a number of polar functional groups. It is generally hydrophobic and is not sufficiently soluble in water or water based solvent mixtures to properly formulate Compound 1 for use in a drug formulation. Compound 1, however, is sufficiently soluble in a variety of organic polar solvents. These organic polar solvents, however, are not typically pharmaceutically acceptable for use in drug formulation.

A nanoemulsion formulation of Compound 1 described below provides a pharmaceutically acceptable solution to the issues of solubility of Compound 1.

In one embodiment, a nanoemulsion formulation of Compound 1 is comprised of a dispersed medium that is suspended in a dispersing medium. The dispersed medium is a solvent capable of dissolving Compound 1. Non-limiting examples of such solvents are non-water miscible organic solvents; oils; glycerides; fats; lipophilic solvents; and mixtures of any two or more of these solvents. Non-limiting examples of a dispersing medium include water; a water miscible solvent; a hydrophilic solvent; and a mixture of any two or more of these solvents.

The nanoemulsion of Compound 1 may comprise other components to improve characteristics of the formulation or of the nanoemulsion. Examples of such other components include surfactants, emulsifying agents; osmotic adjusting agents, antioxidant/stabilizers, pH modification agents; inorganic salts; buffering agents; and combinations or mixtures of them.

Oils used in the nanoemulsion include any one or more vegetable oils, non-limiting, representative examples of which include castor oil, soybean oil, sesame oil, safflower oil, corn oil, coconut oil, linseed oil, mineral oil, olive oil, and peanut oil. Glycerides that may be used in the nanoemulsion formulation include: monoglycerides; diglycerides; triglycerides, which each may be long chain, medium chain, or short chain glycerides or combination thereof. As used herein, “long chain triglycerides” refer to tri-esters of glycerol having a mixture of saturated and unsaturated fatty acids with carbon length of 18 to 24 carbons. While “medium chain triglycerides” refer to tri-esters of glycerol having carbon length of 6 to 12 carbons. “Short chain triglycerides” are fatty acids containing fewer than 6 carbon chain. In a non-limiting example, the oil concentration in the nanoemulsion formulation is in the range of 0.1%-40% by weight. In another non-limiting example, the oil concentration in the nanoemulsion is in the range of 5.0%-30.0% by weight. In another non-limiting example, the oil concentration is in the range of 10.0%-25.0% by weight.

In another embodiment, a nanoemulsion of Compound 1 includes one or more surfactants that reduce interfacial tension and make the droplets more thermodynamically stable in the dispersed phase to reduce any tendency toward aggregation, creaming and droplet growth. Such surfactants may be ionic or non-ionic. Non-limiting examples of surfactants that may be introduced into the nanoemulsion formulation include lecithin, polyethylene glycol (PEG) lipids, Pluronic® F68, and sodium oleate. Lecithin has been used extensively to stabilize injectable nanoemulsions.

In a representative example of a nanoemulsion of Compound 1, lecithin is used both as solubilizer and an emulsifier. Lecithin is a natural phospholipid that can be obtained from soybeans, eggs, sunflower seeds, and others. In a representative example of this embodiment, the lecithin is injectable lecithin, which is free of irritating, inflammatory, and immunogenic agents, examples of which are proteins. The lecithin contains more than 90% by weight phosphatidylcholine (PC), such as Phospholipon 90G (PL90G). The lecithin or surfactant concentration in the nanoemulsion formulation may be in the range of 0.1%-40% by weight. In another example, the surfactant concentration in the nanoemulsion is in the range of 5.0% to 30.0% by weight or, alternatively, in the range of 10.0%-25.0% by weight.

As used herein, the osmotic adjusting agent is an agent that is used in the composition to increase the osmotic pressure of the nanoemulsion of compound 1. The nanoemulsion of Compound 1 according to any one of the preceding embodiments or examples has an osmotic pressure in the range of 100-1000 mOsm, but, alternatively, has an osmotic pressure in the range of 200-400 mOsm or at 300 mOsm. Non-limiting, representative examples of osmotic adjusting agents that can be introduced into the nanoemulsion formulation to increase osmotic pressure include but are not limited to potassium chloride, sodium chloride, sugars such as disaccharides (e.g., sucrose, mannitol, trehalose etc.), glycerol, and mixtures of any two or more of them. A preferred osmotic agent for a nanoemulsion formulation as described herein is sucrose and used in the range of 1% to 20% w/w.

Any of the herein described embodiments of a nanoemulsion of Compound 1, may, optionally, contain one or more antioxidants, a stabilizer, or both to prevent or slow down the oxidation of lipid component (e.g., lecithin and oils) in the nanoemulsion. Any pharmaceutically acceptable stabilizer or antioxidant may be used, non-limiting examples of which are inert gas, dextrose, fructose, cysteine, L-methionine, and metal chelators such as Ethylenediaminetetraacetic acid (EDTA). Each of the disclosed embodiments and examples of the nanoemulsion formulation of Compound 1 may, optionally, include as an antioxidant EDTA calcium disodium in a concentration in the range is 0.001-0.2% by weight or, alternatively, in the range of 0.01-0.06% by weight.

A pH modifier may be added to the nanoemulsion to obtain a desired final pH. pH modifiers that may be added to the formulation include organic and inorganic acids and bases or their conjugate acids and conjugate bases. Nonlimiting of suitable pH modifiers include lactic acid, acetic acid, citric acid, phosphoric acid, arginine, histidine, and glycine. Buffer solutions may also be added to the nanoemulsion formulation to control the resulting pH of the formulation. Non limiting buffers that may be used include citrate, lactate, acetate, histidine, phosphate, and glycine-NaOH buffers. The pH of the nanoemulsion formulation may be in the range of 3.0-10.0. More preferably the pH of the nanoemulsion formulation is in the range of 4.0-9.0 and, even more preferably, is in the range of 4.0-8.0. Even more preferably the pH of the nanoformulation is in the range of 4.0-7.0.

A nanoemulsion formulation of Compound 1 may be made by dissolving Compound 1 in a dispersed medium; dissolving any additional lipophilic components in the dispersed medium; dissolving any water soluble or hydrophilic components in a dispersing medium; mixing together the resulting coarse emulsion; and treating, if necessary, the resulting coarse emulsion to form nanosized emulsion droplets of the dispersed medium within the dispersing medium.

The mixture resulting from mixing the dispersing medium solution and the dispersed medium solution may result in a mixture, a macro emulsion or a nanoemulsion. If the result is a mixture or a macro emulsion, then further treatment of the resulting mixture is required to convert the resulting mixture into a suitable nanoformulation. This may be done by any suitable known nanoemulsifying procedure, such as by microfluidization, ultrasonication, high pressure homogenization, or phase inversion. Any such method may be used to create a nanoemulsion with the desired droplet size. In addition, the resulting mixture of dispersed medium and dispersing medium may be treated by any one or a combination of nanoemulsification methods multiple times to achieve the desired droplet size.

The resulting droplet size of dispersed medium within the dispersed medium in the resulting nanoemulsion may be in the range of 10-500 nm, is preferably in the range of 20-100 nm, and is even more preferable in the range of 40-80 nm.

The ratio of dispersed medium to dispersing medium, by weight, may be in the range of 20:1 to 1:20. More preferably the ratio of dispersed medium to dispersing medium, by volume, is in the range of 2:1 to 1:20 and more preferably in the range of 2:1 to 1:5.

A representative embodiment of the nanoemulsion formulation may be formed as follows. 0.1-3.0% w/w of Compound 1 may be mixed with or dissolved in 5.0-50.0% w/w of a glyceride, such as long chain triglycerides and medium chain triglycerides; and an oil; a fat; or a mixture of the foregoing. If Compound 1 does not fully dissolve in the resulting mixture, then a small amount of polar volatile organic solvent, such as methanol or ethanol, may be added to promote dissolution of Compound 1. Once Compound 1 is dissolved the polar volatile organic solvent may be removed by evaporation. 0.1-30.0% w/w of one or more surfactants or emulsifying agents, such as sucrose, is then added to the resulting solution and mixed thoroughly. 50.0-80.0% w/w of water is then added and mixed thoroughly to create a mixture, macroemulsion, or micro emulsion. The resulting product is then treated by any known nanoemulsion formation methods, such as high shear and microfluidization, until a nanoemulsion with suitable droplet sizes is formed.

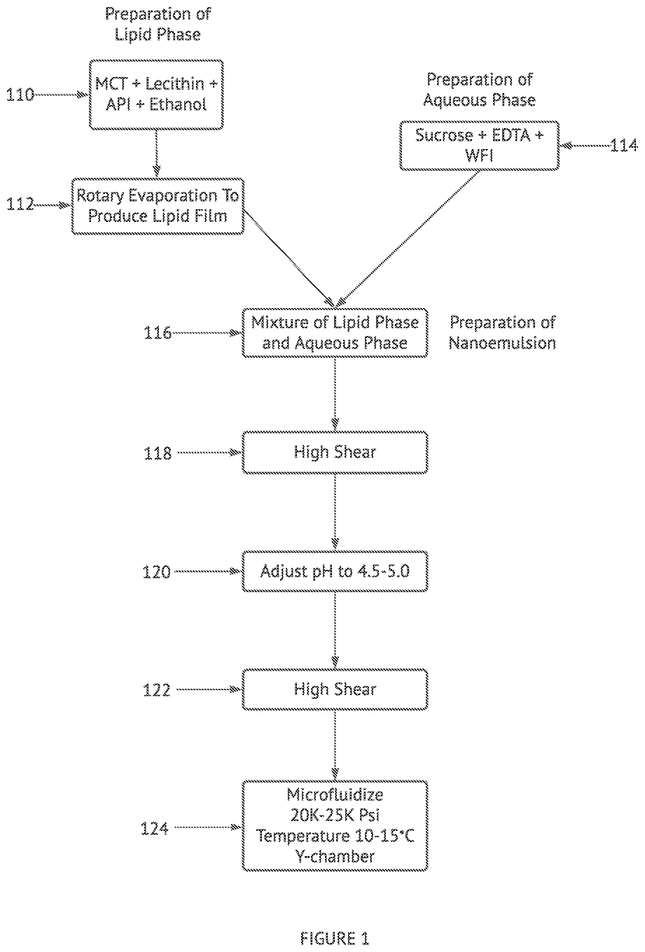

Reference is now made to FIG. 1, which depicts one embodiment of a method of making a nanoemulsion formulation of Compound 1. An emulsion solvent evaporation method was used to manufacture nanoemulsion. The method uses a volatile solvent (e.g., ethanol, chloroform, ethyl acetate and dichloromethane) to solubilize lipid components, which is evaporated by high temperature, vacuum or continuous stirring and the resulted lipid film is then emulsified with aqueous phase. The most pharmaceutically acceptable organic and volatile solvent is ethanol. In step 110, preparation of the lipid phase component of a nanoemulsion is initiated by mixing a medium chain triglyceride (MCT), a lecithin, the active pharmaceutical ingredient (API), and ethanol. In step 112, the resulting solution from step 110 is subjected to rotary evaporation to remove the ethanol to 2% or less by weight, which can be determined by mass comparison. In the embodiment of FIG. 1, the resulting product is a lipid film containing the active pharmaceutical ingredient. The aqueous phase of the nanoemulsion is prepared in step 114, where sucrose, EDTA, and water for injection are mixed to create an aqueous solution. In step 116, the preparation of the nanoemulsion formulation is initiated by mixing the lipid film resulting from step 112 with the aqueous solution produced by step 114. The resulting mixture of step 116 is subjected to high shear to form a uniform mixture or an emulsion and to further disperse the lipid phase within the aqueous phase. In step 120, the pH of the resulting emulsion is adjusted with a suitable pH adjusting agent (i.e., 5N sodium hydroxide). Preferably the resulting pH is in the range of 4.5-5.0, more preferably the resulting pH of the mixture is in the range of 4.8-5.0. In step 122, the pH adjusted emulsion of step 120 is further subjected to high shear to reduce emulsion droplet size and to refine nanoemulsion characteristics. High shear treatment of step 120 is continued until emulsion droplet size is preferably in the range of 300-500 nm. The resulting emulsion from step 124 is subjected to microfluidization to further reduce emulsion droplet size. Preferably microfluidization is performed at 20,000-25,000 Psi at 10 to 15° C., using a Y-chamber. Microfluidization is continued until the droplet size of the emulsion is reduced to the desired size. Preferably microfluidization is continued until the droplet size is reduce to the range of 50-150 nm.

Several example formulations were prepared. In the following example descriptions: The therapeutic agent is Compound 1. LCT is a USP grade long chain triglyceride, such as that available from CRODA [Super refined soybean oil, SR49571, CRODA, NJ, USA]. MCT is a USP grade medium chain triglyceride or miglyol 812, such as that available from RN Oleochemical [Witten, Germany]. Phospholipon 90G (PL90G) is an injectable grade lecithin such as that available from LIPOID [Newark, NJ, USA]. SWFI is USP grade sterile water for injection such as that available from OmniPur [Cat. No. 4.86505, Millipore Sigma, Burlington, Massachusetts, USA]. The sucrose used in the following formulation examples was USP grade sucrose, such as that available from Spectrum. Capsul MCM used from Abitec. Poloxamer 188/F68 used from BASF.

Example 1: A 40 g batch of nanoemulsion formulation of the therapeutic agent was formed from the following components: 0.2 g therapeutic agent, 4 g LCT, 4 g MCT, 4 g PL90G, 8 g sucrose, and 19.8 g SWFI. The LCT, MCT, PL90G, and therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Removal of the ethanol was continued by rotary evaporation until residual ethanol was reduced to ≤2.0%, which can be verified by weight comparison. Removal of ethanol resulted in a clear and transparent lipid phase. At this point, a sucrose solution in SWFI prepared separately was then added to reconstitute the lipid phase. The remaining SWFI was then added to bring the total weight of the formulation to 40 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 0.5%, LCT 10%, MCT 10%, PL90G 10%, sucrose 20%, SWFI 49.5%.

Example 2: Another example embodiment of 40 g batch of nanoemulsion formulation was prepared as follows: 2 g LCT, 2 g MCT, 4 g PL90G, and 0.2 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Removal of the ethanol was continued by rotary evaporation until residual ethanol was reduced to ≤2.0%, which can be verified by weight comparison. Removal of ethanol resulted in a clear lipid phase. 8 g sucrose was used to separately prepare a sucrose solution in SWFI and then added to reconstitute the lipid phase. The remaining SWFI was added to bring to total weight of the formulation to 40 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 0.5%, LCT 5%, MCT 5%, PL90G 10%, sucrose 20%, SWFI 59.5%.

Example 3: Another example embodiment formulation was prepared as follows: 4 g MCT, 4 g PL90G, and 0.2 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Removal of the ethanol was continued by rotary evaporation until residual ethanol was reduced to ≤2.0%, which can be verified by weight comparison. Removal of ethanol resulted in a clear lipid phase. 8 g sucrose was used to separately prepare a sucrose solution in SWFI and then added to reconstitute the lipid phase. The remaining SWFI was added to bring to total weight of the formulation to 40 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 0.5%, MCT 10%, PL90G 10%, sucrose 20%, and SWFI 59.5%.

The resulting formulations of examples 1-3 were tested to determine pH, clarity, droplet size, seta potential, assay, impurities, and dilutability in WFI or NS. The results are as summarized in Table 1.

TABLE 1

| Property | Example 1 | | Example 2 | | Example 3 | |
|---|---|---|---|---|---|---|
| Appearance | Cloudy | | Cloudy to | | Cloudy to | |
| | | | Semi-transparent | | Semi-transparent | |
| pH | 6.06 | | 5.85 | | 5.75 | |
| Droplet Size | 330 | nm | 119 | nm | 134 | nm |
| Zeta Potential | −2.87 | mV | +11.7 | mV | +11.1 | mV |
| Therapeutic | 5.09 | mg/mL | 5.43 | mg/mL | 4.57 | mg/mL |
| Assay | | | | | | |
| Filterability | No | | Yes | | Yes | |
| at 0.2 μm | | | | | | |

| | | | |
|---|---|---|---|
| Dilutability with | Yes | Yes | Yes |
| WFI/NS | | | |

Example 4: Another example embodiment formulation was prepared as follows: 2 g LCT, 2 g Capmul MCM, 4 g PL90G, and 0.2 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Complete removal of the ethanol can be verified by weight comparison. Removal of ethanol resulted in a clear lipid phase. 8 g sucrose was used to separately prepare a sucrose solution in SWFI and then added to reconstitute the lipid phase. The remaining SWFI was added to bring to total weight of the formulation to 40 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 0.5%, LCT 5%, Capmul MCM 5%, PL90G 10%, sucrose 20%, SWFI 59.5%.

Example 5: Another example embodiment formulation was prepared as follows: 2 g LCT, 2 g MCT, 4 g PL90G, and 0.6 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Complete removal of the ethanol can be verified by weight comparison. Removal of ethanol resulted in a clear lipid phase. 8 g sucrose was used to separately prepare a sucrose solution in SWFI which was then added to reconstitute the lipid phase. The remaining SWFI was added to bring to total weight of the formulation to 40 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 1.5%, LCT 5%, MCT 5%, PL90G 10%, sucrose 20%, SWFI 59.5%.

The resulting formulation of example 5 had the properties as listed in Table 2.

TABLE 2

| Property | Example 5 | |
|---|---|---|
| Appearance | Cloudy | |
| pH | 6.05 | |
| Droplet Size | 240 | nm |
| Zeta Potential | +9.33 | mV |
| Therapeutic Assay Pre-Filter | 14.83 | mg/mL |
| Therapeutic Assay Post-Filter | 12.46 | mg/mL |

| | |
|---|---|
| Filterability at 0.2 μm | No |

Example 6: Another example embodiment formulation was prepared as follows: 1.6 g MCT, 1.6 PL90G, and 0.2 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Complete removal of the ethanol can be verified by weight comparison. Removal of ethanol resulted in a clear lipid phase. 3.6 g sucrose dissolved in SWFI was then added to reconstitute the lipid phase. SWFI was added to bring to total weight of the formulation to 20 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 1.0%, MCT 8%, PL90G 8%, sucrose 18%, SWFI 65%.

The resulting formulation of example 6 had the properties as listed in Table 3.

TABLE 3

| Property | Example 6 | |
|---|---|---|
| Appearance | Semi-transparent | |
| pH | 5.09 | |
| Droplet Size | 70 | nm |
| Zeta Potential | +38 | mV |
| Therapeutic Assay Pre-Filter | 10.0 | mg/mL |
| Therapeutic Assay Post-Filter | 10.7 | mg/mL |
| Filterability at 0.2 μm | Yes | |
| Dilutability with WFI/NS | Yes | |
| PFAT | 0.001% | |
| Osmolarity | 690 | mOsm |

Example 7: Another example embodiment formulation was prepared as follows: 1.6 g MCT, 1.6 g PL90G, and 0.2 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Complete removal of the ethanol can be verified by weight comparison. Removal of ethanol resulted in a clear lipid phase.

2.4 g sucrose in SWFI was then added to reconstitute the lipid phase. SWFI was added to bring to total weight of the formulation to 20 g. The resulting formulation was high sheared to reduce droplet size to <500 nanometers. The resulting formulation was then microfluidized with 5 passes to produce a droplet size of <100 nanometers. The resulting formulation had the following component percentages (% w/v): therapeutic agent 1.0%, MCT 8%, PL90G 8%, sucrose 12%, SWFI 71%.

The resulting formulation of example 6 had the properties as listed in Table 4.

TABLE 4

| Property | Example 6 | |
|---|---|---|
| Appearance | Semi-transparent | |
| pH | 5.36 | |
| Droplet Size | 83 | nm |
| Zeta Potential | +8.54 | mV |
| Therapeutic Assay Pre-Filter | 9.04 | mg/mL |
| Therapeutic Assay Post-Filter | 8.37 | mg/mL |
| Filterability at 0.2 μm | Yes | |
| Dilutability with WFI/NS | Yes | |
| Osmolarity | 518 | mOsm |

Example 8: Another example embodiment formulation was prepared as follows: 1.6 g MCT, 0.32 g Poloxamer 188, 1.28 g PL90G, and 0.2 g therapeutic agent were added to a 100 mL round bottom flask. The mixture was stirred until dissolved, and a clear solution was obtained. If upon mixing, the mixture remained cloudy, a sufficient amount of ethanol was added until the mixture was converted to a clear solution. If addition of ethanol was necessary, the ethanol was removed from the mixture by rotavapor evaporation. Complete removal of the ethanol can be verified by weight comparison. Removal of ethanol resulted in a clear lipid phase. 1.6 g sucrose in SWFI was then added to reconstitute the lipid phase. SWFI was added to bring to total weight of the formulation to 20 g. The resulting formulation was high sheared to reduce droplet size to <500 nanometers. The resulting formulation was then microfluidized with passes to produce a droplet size of <100 nanometers. The resulting formulation had the following component percentages (% w/v): therapeutic agent 1.0%, MCT 8%, PL90G 8%, sucrose 12%, SWFI 71%.

The resulting formulation of example 6 had the properties as listed in Table 5.

TABLE 5

| Property | Example 6 | |
|---|---|---|
| Appearance | Semi-transparent | |
| pH | 5.24 | |
| Droplet Size | 73 | nm |
| Zeta Potential | +17 | mV |
| Therapeutic Assay Pre-Filter | 8.83 | mg/mL |
| Therapeutic Assay Post-Filter | 7.41 | mg/mL |
| Filterability at 0.2 μm | Yes | |
| Osmolarity | 314 | mOsm |

Examples 9-15: Example formulations 9-15 were prepared as described below, with the components as listed in Table 6. MCT, PL90G, and the therapeutic agent of FIG. 1 were added to a 100 mL round bottom flask and mixed until dissolved. If mixing these components resulted in an unclear suspension, ethanol was added until all components dissolved, and the solution was clear. If ethanol was added, the ethanol was removed by rotary evaporation to form a clear phase liquid. Sucrose with or without poloxamer 188 or glycerin in SWFI was added to reconstitute the lipid phase. The resulting formulation was further diluted with SWFI to provide a total weight of 20 grams. The resulting formulation was subject to high shear to reduce droplet size to <500 nm. The resulting formulation was microfluidized with five passes to reduce droplet size to <100 nm.

TABLE 6

| | Example | Example | Example | Example | Example | Example | Example |
|---|---|---|---|---|---|---|---|
| Property | 9 | 10 | 11 | 12 | 13 | 14 | 15 |
| Compound 1 | 0.5 | 0.5 | 0.5 | 1.0 | 0.75 | 1.0 | 0.85 |
| MCT (grams) | 8 | 8 | 8 | 15 | 8 | 8 | 8 |
| Poloxamer 188 | 1.6 | 0 | 0 | 0 | 0 | 0 | 0 |
| (grams) | | | | | | | |
| PL90G (grams) | 6.4 | 8 | 8 | 8 | 8 | 8 | 8 |
| Glycerin (grams) | 0 | 0 | 5 | 0 | 0 | 0 | 0 |
| Sucrose (grams) | 12 | 12 | 0 | 12 | 10 | 10 | 10 |
| SWFI (grams) | 71.5 | 71.5 | 78.5 | 64 | 73.25 | 73 | 73.15 |

The resulting formulation of examples 9-15 had the properties as listed in Table 7.

TABLE 7

| | Example | | Example | | Example | | Example | | Example | | Example | | Example | |
|---|---|---|---|---|---|---|---|---|---|---|---|---|---|---|
| Property | 9 | | 10 | | 11 | | 12 | | 13 | | 14 | | 15 | |
| Appearance | ST* | | ST* | | ST* | | ST* | | ST* | | ST* | | ST* | |
| pH | 5.50 | | 5.20 | | 4.94 | | 4.56 | | 5.34 | | 5.45 | | 5.00 | |
| Droplet Size | 72 | nm | 61 | nm | 60 | nm | 54 | nm | 107 | nm | 80 | nm | 68 | nm |
| Zeta Potential | +13.8 | mV | +23.1 | mV | +26 | mV | +48 | mV | +25 | mV | +26 | mV | +20.3 | mV |
| Therapeutic | 4.81 | mg/mL | 4.5 | mg/mL | 4.65 | mg/mL | 9.49 | mg/mL | 7.95 | mg/mL | 9.12 | mg/mL | 7.73 | mg/mL |
| Assay | | | | | | | | | | | | | | |
| Pre-Filter | | | | | | | | | | | | | | |
| Therapeutic | 4.86 | mg/mL | 4.5 | mg/mL | 4.68 | mg/mL | 9.42 | mg/mL | 7.73 | mg/mL | 8.83 | mg/mL | 7.73 | mg/mL |
| Assay | | | | | | | | | | | | | | |
| Post-Filter | | | | | | | | | | | | | | |
| Filterability at | Yes | | Yes | | Yes | | Yes | | Yes | | Yes | | Yes | |
| 0.2 μm | | | | | | | | | | | | | | |
| Osmolarity | 424 | mOsm | 452 | mOsm | 696 | mOsm | 474 | mOsm | 425 | mOsm | 490 | mOsm | 382 | mOsm |

*ST: Semi-Transparent

Physical and chemical stability of Compound 1 was tested in nanoemulsion formulation. Batches of nanoemulsions were prepared at 5 mg/mL and 7.5 mg/mL of Compound 1, labeled Batch A and Batch B, respectively, with the components as listed in Table 8.

TABLE 8

| Components (% w/w) | Batch A | Batch B |
|---|---|---|
| Compound 1 | 0.5 | 0.75 |
| MCT | 8 | 8 |
| Soy Lecithin (PL90G) | 8 | 8 |
| Sucrose | 10 | 10 |
| SWFI | 73.5 | 73.25 |
| Total | 100 | 100 |

The resulting nanoemulsions were passed through a 0.2-micron sterile filter to sterilize, filled into glass vials (2 mL/vial) and crimp sealed. The sealed vials were stored at −20° C., 2 to 8° C., and 25° C. Individual vials were pulled at 0-days, 2-week, 1-month, 3-month, and 6-month time points and tested for appearance, pH, clarity, particulate matter, assay, and impurities of Compound 1. The stability results are summarized in the following Table 9.

TABLE 9

| | | Storage | Day 0 | | 2 wks | | 1 mo | | 3 mo | | 6 mo | |
|---|---|---|---|---|---|---|---|---|---|---|---|---|
| | Proposed | Temperature | Batch | Batch | Batch | Batch | Batch | Batch | Batch | Batch | Batch | Batch |
| Test | Specs | (° C.) | A | B | A | B | A | B | A | B | A | B |
| Appearance | Semi- | −20° C. | Pass | | SC* | SC* | SC* | SC* | SC | SC* | SC* | SC* |
| | transparent | 2 to 8° C. | | | Pass | Pass | Pass | Pass | Pass | Pass | Pass | Pass |
| | emulsion | 25° C. | | | SC* | SC* | SC* | SC* | SC* | SC* | SC* | SC* |
| Assay (mg/mL) | Report | −20° C. | 4.96 | 7.32 | 4.89 | 7.55 | 4.92 | 7.49 | 4.89 | 7.02 | 4.86 | 8.00 |
| | | 2 to 8° C. | | | 5.14 | 7.30 | 4.78 | 7.29 | 4.04 | 6.82 | 3.19 | 7.31 |
| | | 25° C. | | | 4.45 | 7.31 | 4.08 | 7.21 | 2.70 | 7.02 | 1.14 | 6.65 |
| % Initial | 90.0% to | −20° C. | 100 | | 99 | 103 | 99 | 102 | 99 | 96 | 98 | 109 |
| (over T0) | 110.0% of | 2 to 8° C. | | | 104 | 100 | 96 | 100 | 81 | 93 | 64 | 100 |
| | label claim | 25° C. | | | 90 | 100 | 82 | 99 | 54 | 96 | 23 | 91 |
| Total increase | Report | −20° C. | 0 | 0 | 0 | 0 | 0 | 0 | 0 | 0 | 0 | 0 |
| of impurity (% | | 2 to 8° C. | | | 0.66 | 0 | 1.35 | 0 | 6.91 | 0 | 32.59 | 0.45 |
| peak area over | | 25° C. | | | 6.85 | 0 | 15.45 | 0 | 32.78 | 0 | 71.6 | 12.3 |
| API peak area) | | | | | | | | | | | | |
| Zeta potential | 10-30 mV | −20° C. | 19 | 19 | 25 | 31 | 31 | 31 | 29 | 29 | 39 | 37 |
| (mV) | | 2 to 8° C. | | | 24 | 20 | 28 | 21 | 21 | 23 | 24 | 20 |
| | | 25° C. | | | 20 | 21 | 26 | 26 | 26 | 24 | 28 | 29 |
| Droplet | Average: | −20° C. | 58 | 80 | 150 | 165 | 164 | 197 | 223 | 151 | 209 | 131 |
| Diameter (nm) | 50-150 nm | 2 to 8° C. | | | 66 | 98 | 70 | 105 | 69 | 99 | 66 | 100 |
| | | 25° C. | | | 77 | 143 | 80 | 153 | 114 | 169 | 135 | 171 |

*SC: Slight Cloudy

The stability results indicated that nanoemulsion formulations of Compound 1, as described herein, are chemically stable over 6 months.

Examples 16 and 17. Two additional examples of the nanoemulsion formulation according to the procedures described above with the components as indicated in Table 10.

TABLE 10

| Components (% w/w) | Example 16 | Example 17 |
|---|---|---|
| Compound 1 | 0.73 | 0.70 |
| MCT | 8 | 8 |
| Soy Lecithin (PL90G) | 8 | 8 |
| Sucrose | 10 | 18 |
| EDTA Na₂H₂O | 0.06 | 0.06 |
| SWFI | 73.21 | 65.24 |
| Total | 100 | 100 |

Although the invention has been described with reference to specific embodiments, these descriptions are not meant to be construed in a limiting sense. Various modifications of the disclosed embodiments, as well as alternative embodiments of the invention, will become apparent to persons skilled in the art upon reference to the description of the invention. It should be appreciated by those skilled in the art that the conception and the specific embodiment disclosed might be readily utilized as a basis for modifying or designing other structures for carrying out the same purposes of the present invention. It should also be realized by those skilled in the art that such equivalent constructions do not depart from the spirit and scope of the invention as set forth in the appended claims. It is therefore contemplated that the claims will cover any such modifications or embodiments that fall within the true scope of the invention.

1. A formulation comprising:
compound 1 or a pharmaceutically acceptable salt of compound 1, wherein compound 1 has the molecular structure of 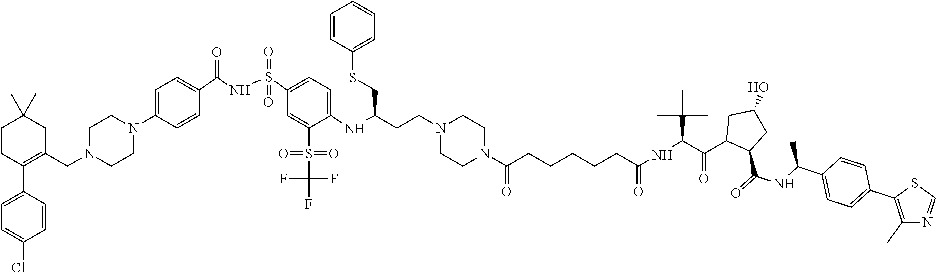 [SMILES: Cc1ncsc1-c1ccc([C@H](C)NC(=O)[C@@H]2C[C@@H](O)CC2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1]
a dispersed medium; and
a dispersing medium.

2. The formulation of claim 1, wherein the formulation is an emulsion.

3. The formulation of claim 2, wherein the formulation is a nanoemulsion.

4. The formulation of any proceeding claim, in which the formulation further comprises one or more: a surfactant, an emulsifying agent; an osmotic adjusting agent, an antioxidant, a stabilizer, a pH modification agent, an inorganic salt, or a buffering agent.

5. The formulation of claim 4, wherein the surfactant is lecithin, polyethylene glycol (PEG) lipids, Pluronic® F68, or sodium oleate.

6. The formulation of claim 4, wherein the osmotic agent is potassium chloride, sodium chloride, a sugar, or glycerol.

7. The formulation of claim 4, wherein the antioxidant is an inert gas, dextrose, fructose, cysteine, L-methionine, a metal chelator, ethylenediaminetetraacetic acid (EDTA), or ethylenediaminetetraacetic acid calcium disodium.

8. The compound of claim 4, wherein the pH modification agent is lactic acid, acetic acid, citric acid, phosphoric acid, arginine, histidine, glycine, or a buffer solution.

9. The formulation of any proceeding claim, wherein the dispersed medium is a water miscible organic solvent, an oil, a glyceride, a fat, a lipophilic solvent, or a mixture of two or more of a water miscible organic solvent, an oil, a glyceride, a fat, and a lipophilic solvent.

10. The formulation of any proceeding claim, wherein the dispersing medium is water, a water miscible solvent, a hydrophilic solvent, or a mixture of two or more of water, a water miscible solvent, and a hydrophilic solvent.

11. The formulation of any proceeding claim, wherein the dispersed medium is long chain triglyceride, a medium chain triglyceride, or a short chain triglyceride.

12. The formulation of any proceeding claim, comprising:
0.5-2.0% w/v of compound 1;
0-10% w/v of a long chain triglyceride;
0-10% w/v of a medium chain triglyceride;
5-10% w/v of a lecithin; and
5-20% w/lv of a sugar.

13. The formulation of claim 12, comprising:
0.5% w/v of compound 1;
10% w/v of a long chain triglyceride;
10% w/v of a medium chain triglyceride;
10% w/v of PL90G; and
20% w/v of a sucrose.

14. The formulation of claim 12, comprising:
0.7% w/w of compound 1;
8% w/w of a medium chain triglyceride;
8% w/w of PL90G;
18% w/w of a sucrose; and
0.6% w/w EDTA Na₂H₂O.

15. The formulation of claim 12, comprising:
0.5-1.0% w/w of compound 1;
−10% w/w of a medium chain triglyceride; −10% w/w of PL90G;
0-20% w/w of a sucrose; and
0.01-2.0% w/w EDTA Na₂H₂O.
